(19) United States
(12) Patent Application Publication
(10) Pub. No.: US 2014/0314729 A1
(43) Pub. Date: Oct. 23, 2014

(54) NUTRACEUTICAL FORMULATION FOR TREATMENT OF ELEVATED CHOLESTEROL AND CARDIOVASCULAR DISEASE

(71) Applicant: Creative Medical Health Inc., Phoenix, AZ (US)

(72) Inventor: Amit Patel, Salt Lake City, UT (US)

(21) Appl. No.: 14/251,005

(22) Filed: Apr. 11, 2014

Related U.S. Application Data
(60) Provisional application No. 61/811,694, filed on Apr. 12, 2013.

(51) Int. Cl. A61K 36/9068; A61K 31/455; A61K 36/328; A61K 36/88; A61K 36/28; A61K 36/899; A61K 36/53; A61K 31/205; A61K 31/4402; A61K 31/122; A61K 31/197; A61K 36/87; A61K 36/42; A61K 36/38; A23L 1/30; A23L 1/302; A61K 31/047

(52) U.S. Cl. CPC A61K 36/9068; A61K 31/047; A61K 31/455; A61K 36/328; A61K 36/88; A61K 36/28; A61K 36/899; A61K 36/53; A61K 31/205; A61K 31/4402; A61K 31/122; A61K 31/197; A61K 36/87; A61K 36/42; A61K 36/38; A23L 1/3002; A23L 1/302
USPC 424/94.1; 514/690

(57) ABSTRACT

Disclosed are compositions of matter useful for the treatment of elevated blood cholesterol. In one embodiment a nutraceutical composition is administered to a patient in need of said composition comprising of the following combination of ingredients: policosanol, niacin, guggul, garlic, cynara scolymus, red yeast rice, ginger, holy basil, L-carnitine, chromium picolinate, coenzyme Q10, pantothenic acid, grape seed extract, momordica charantia, and garcinia indica.

### CROSS REFERENCE TO RELATED APPLICATIONS

This application claims priority to Provisional Application Ser. No. 61/811,694 filed Apr. 12, 2013, and entitled “Cholesterol-lowering herbal formulation”, which is hereby expressly incorporated by reference in its entirety.

### FIELD OF THE INVENTION

The present technology pertains to the field of cholesterol management. Specifically, the invention relates to the use of natural compounds for prevention and management of elevated blood cholesterol. More specifically, the invention relates to the use of combinations of policosanol, niacin, guggul, garlic, cynara scolymus, red yeast rice, ginger, holy basil, L-carnitine, chromium picolinate, coenzyme Q10, pantothenic acid, grape seed extract, momordica charantia, and garcinia indica.

### BACKGROUND

Cholesterol is involved in many vital physiological processes, such as maintaining membrane integrity of eukaryotic cells, manufacturing vitamin D on the skin, synthesis of steroid hormones, and formation of neural synapses in the brain [1]. However, abnormally elevated cholesterol levels in the blood can lead to health consequences; specifically coronary heart disease (CHD), one of the leading causes of death worldwide. Atherosclerosis is the pathological process that typically underlies CHD morbidity and mortality. This process involves formation of plaques in the intima and media of the arterial wall. These atherosclerotic lesions result from the progressive accumulation of cholesterol and lipids, extracellular matrix material, and inflammatory cells along the arterial walls (reviewed in [2]).

The concentrations of the various types of cholesterol in the blood are predictive of the risk of atherosclerosis and coronary heart disease. Low-density lipoprotein (LDL) cholesterol particles are lipoproteins that enable transport of fat molecules in the bloodstream. An LDL particle consists of an apolipoprotein B-100 molecule and a hydrophobic core comprised mainly of esterified cholesterol. LDL represents the so-called “bad” cholesterol that is associated with the risk of cardiovascular disease. Like LDL cholesterol, very low-density lipoprotein (VLDL) cholesterol levels correlate with a risk of CHD. VLDL contain 62% triglycerides, 12% cholesterol, and apolipoprotein B100, E and C. VLDL particles are produced in the liver and serve to carry triglycerides to peripheral tissues in the fasting state, and the VLDL remnants are modified and converted to LDL by hepatic lipase. In contrast, high-density lipoprotein (HDL) cholesterol particles are comprised of 1-2% triglycerides, 30-36% cholesterol, and apolipoprotein A, E and C. HDL is the so-called “good” cholesterol that functions to aid clearance or inhibit uptake of LDL cholesterol. HLD particles bind unesterified cholesterol released from the tissues and deliver cholesterol back to the liver for processing, which is referred to as reverse cholesterol transport.

Elevated levels of low-density lipoprotein cholesterol (LDL) and triglycerides (TC) represent risk factors for CHD, whereas high concentrations of plasma high-density lipoprotein cholesterol (HDL) are considered healthy and protective against CHD. The cholesterol content per LDL molecule can exhibit a large variation between individuals; therefore, LDL particle size and number can provide independent measures of the risk of CHD [3]. The general guidelines according to the American Heart Association state that healthy cholesterol levels in blood lipoprotein profiles are <200 mg/dL for total cholesterol, 60 mg/dL and above for HDL cholesterol, less than 100 mg/dL for LDL cholesterol, and less than 150 mg/dL for triglycerides (http://www.heartorg/HEARTORG/Conditions/Cholesterol/AboutCholesterol/What-Your-Cholesterol-Levels-Mean_UCM_{—}305562_Article.jsp).

Cholesterol homeostasis is achieved by de novo synthesis in the body and by absorption in the gastrointestinal tract from dietary sources. Mammalian cells increase cholesterol levels through de novo synthesis, primarily by hepatocytes in the liver. The liver regulates cholesterol levels in the body by exporting newly synthesized cholesterol to other cells, but also by converting it to bile salts for removal from the body. Dietary cholesterol and triglycerides are obtained from foods derived from animal sources, including meats, eggs and dairy products, which account for approximately 20% of the body's total cholesterol content. Absorption of dietary cholesterol occurs within the lumen of the small intestine. Micellar particles in the gallbladder, which are necessary for the digestion and absorption of fat, are released into the lumen of the small intestine in response to ingestion food. Dietary lipids are assimilated into the micelles, which transport the lipids to the plasma membrane of enterocytes that form the lining of the small intestinal lumen. Fatty acids and monoglycerides are taken up into enterocytes and enzymatically processed into triglycerides, while cholesterol uptake is facilitated by the protein Nieman-Pick C1 Like 1 (NPC1L1). A portion of the cholesterol that is taken up is pumped back into the lumen of the intestine by a heterodimer of the ATP-binding cassette transporters, ABCG5 and ABCG8. This action constitutes a mechanism of reducing intestinal cholesterol absorption and promoting cholesterol excretion. The triglycerides and cholesterol that have been taken up by enterocytes are combined with apolipoprotein B-48 into chylomicrons, which carry absorbed dietary fats and cholesterol in the circulation. Rates of cholesterol absorption vary widely among individuals.

Several treatments for elevated cholesterol are known in the art. Cholesterol-modifying regimens include drugs and nutritionally balanced diets that are low in saturated fat and cholesterol. Statins are the most commonly administered medications prescribed for lowering the concentrations of LDL cholesterol in the blood. Statins function by reducing the rate of intracellular cholesterol synthesis through inhibition of 3-hydroxy-3-methylglutaryl (HMG)-CoA reductase, the rate-limiting step in cholesterol biosynthesis in the liver. A number of statins are on the market: atorvastatin (Lipitor and Torvast), fluvastatin (Lescol), lovastatin (Mevacor, Altocor, Altoprev), pitavastatin (Livalo, Pitava), pravastatin (Pravachol, Selektine, Lipostat), rosuvastatin (Crestor) and simvastatin (Zocor, Lipex). In clinical practice, lifestyle modifications consisting of a cholesterol lowering diet and exercise is often recommended before statins are prescribed.

Cholesterol absorption inhibitors slow the absorption of dietary cholesterol, but more important, they also reduce the reabsorption of biliary cholesterol, which accounts for most of the cholesterol in the intestine. The available cholesterol absorption inhibitors are plant sterols/stanols and ezetimibe. Plant sterols/stanols are naturally occurring molecules that diminish dietary cholesterol absorption within the intestinal lumen. They are close molecular mimics of cholesterol, but are much more hydrophobic, thereby allowing them to compete effectively with cholesterol for incorporation into micelles [4]. As a result, they displace cholesterol from micelles [4]. Additionally, when plant sterols are taken up by enterocytes, they are pumped back into the lumen by the action of ABCG5 and ABCG8, which is an effective means for preventing plant sterols from entering the body. In one published study, daily consumption of 1-2 grams of plant sterols or stanols was shown to cause 10-20% reduction in LDL cholesterol [5]. Ezetimibe also lowers cholesterol blood cholesterol by binding to the NPC1L1 protein on the gastrointestinal tract epithelial cells and hepatocytes, and reducing cholesterol uptake [6]. Moreover, decreased absorption of cholesterol has secondary effects, leading to upregulation of LDL receptors on the surfaces of cells and increased LDL uptake by cells. Overall, these events lead to a decrease in LDL levels in the blood that could contribute to atherosclerosis.

It is known in the art that HLD levels can be increased and LDL levels can be reduced by aerobic exercise and dietary modifications. Changes in the quality of fat consumed can be achieved by replacing saturated fat with unsaturated fat in order to reduce the systemic concentrations of LDL choleseterol and increase HDL cholesterol. In recent years, nutraceuticals and functional foods have attracted interest as possible alternative therapies for lowering plasma TC, including for patients who present with marginally elevated blood cholesterol concentrations that are not high enough to indicate the necessity for cholesterol-lowering medications. Nutraceuticals and functional foods that lower TC must affect the genes that regulate cholesterol homeostasis. In general, cholesterol-lowering functional foods and nutraceuticals can be classified into several types; namely, intestinal Niemann-Pick C1 like 1 (NPC1L1) competitors, intestinal acyl-CoA:cholesterol acyltransferase 2 (ACAT2) inhibitors, 3-hydroxy-3-methylglutaryl (HMG-CoA) reductase inhibitors, LDL receptor up-regulators, bile acid reabsorption inhibitors, cholesterol-7α-hydroxylase (CYP7A1) activators, and plasma cholesteryl ester transporting protein (CETP) inhibitors. The high cost of long-term treatment with cholesterol-lowering drugs and the requirement for physician monitoring is an impetus for self-directed, safe and effective means for patients to maintain or lower their blood cholesterol levels. Disclosed herein is a combination of natural ingredients for use in favorably affecting cholesterol levels.

### SUMMARY

Embodiments herein relate to a composition for preventing and managing elevated blood cholesterol and its effects in a mammal comprising: policosanol, niacin (vitamin B3), guggul, garlic, cynara scolymus, red yeast rice, ginger, holy basil, L-carnitine, chromium picolinate, coenzyme Q10, pantothenic acid, grape seed extract, and momordica charantia, and garcinia indica in an amount sufficient to ameliorate elevated blood cholesterol in said mammal.

Further embodiments relate to a method for lowering elevated levels of blood cholesterol in a mammal, said method comprising: identifying a mammal suffering from elevated levels of harmful cholesterol; administering a nutraceutical formulation of naturally occurring substances comprising: policosanol, niacin, guggul, garlic, cynara scolymus, red yeast rice, ginger, holy basil, L-carnitine, chromium picolinate, coenzyme Q10, pantothenic acid, grape seed extract, momordica charantia, and garcinia indica in an amount sufficient to ameliorate elevated blood cholesterol in said mammal.

### DETAILED DESCRIPTION

This invention teaches compositions of natural products for the management and prevention of elevated blood cholesterol. One of skill in the art utilizes said formulation as a monotherapy or in conjunction with treatment known to lower the concentrations of harmful forms of cholesterol in the blood. The nutraceutical formulation comprises ingredients selected from the following group: policosanol, niacin, guggul, garlic, cynara scolymus, red yeast rice, ginger, holy basil, L-carnitine, chromium picolinate, coenzyme Q10, pantothenic acid, grape seed extract, mormodica charantia and garcinia indica.

The therapeutic properties of the various components of the composition have been previously described, however, utilization of these compositions in combination for managing and preventing high cholesterol and its associated complications has not been reported. In the current invention, therapeutic compositions are associated with additive/synergistic effects of the named ingredients to manage and prevent elevated levels of harmful forms of cholesterol, as well as to reduce oxidative stress and to reduce atherogenic and cardiovascular complications in mammals.

In one embodiment, policosanol is administered to a mammal along with said composition of ingredients comprising niacin, guggul, garlic, cynara scolymus, red yeast rice, ginger, holy basil, L-carnitine, chromium picolinate, coenzyme Q10, pantothenic acid, grape seed extract, mormodica charantia and garcinia indica. Policosanol is a derivative of sugar cane; specifically, a natural mix of plant alcohols made from the purified wax of sugar-cane leaves. Policosanol is known in the art to improve blood lipid profiles with numerous clinical trials supporting its efficacy in preventing the build-up of harmful cholesterol in the body, while also being safe and tolerable in patients with hypercholesterolemia. In a published meta-analysis involving a total of 4596 patients, policosanol was found to be highly effective at reducing the LDL and triglycerides and increasing the HDL in patients, almost to the same level as drug treatments [7]. Policosanol has also been demonstrated to be effective, when used in combination with other natural substances, in a study of children with familial hypercholesterolemic [8]. For example, in one study, a dietary supplement containing 200 mg red yeast rice extract and 10 mg policosanols was administered once daily and placebo for 8 weeks, separated by a 4-week washout period. The results showed an 18.5% reduction of total cholesterol and a 25.1% reduction in LDL cholesterol.

In another embodiment, niacin (vitamin B3, nicotinic acid) is administered to a mammal in need of a cholesterol-lowering treatment along with said composition of ingredients comprising policosanol, guggul, garlic, cynara scolymus, red yeast rice, ginger, holy basil, L-carnitine, chromium picolinate, coenzyme Q10, pantothenic acid, grape seed extract, mormodica charantia and garcinia indica. Niacin binds to and stimulates a G-protein-coupled receptor, GPR109A, which inhibits fat breakdown in adipose tissue and thereby reduces the concentrations of free fatty acid in the blood [9]. As a consequence, LDL and cholesterol secretion by the liver is reduced. Results from a recent meta-analysis have concluded that niacin supplements either alone or with other cholesterol-modifying agents, reduces the incidence of cardiovascular events [10]. Another study revealed that niacin improves the outcomes in patients undergoing treatment with statins, as measured by lipid profiles [11]. Collectively, the published literature demonstrates that niacin alone or together with cholesterol-lowering drugs reduces the progression of atherosclerosis.

In another embodiment, guggul is administered to a mammal along with a composition of ingredients comprising policosanol, niacin, garlic, cynara scolymus, red yeast rice, ginger, holy basil, L-carnitine, chromium picolinate, coenzyme Q10, pantothenic acid, grape seed extract, mormodica charantia and garcinia indica. Guggul (Commiphora mukul), a flowering plant that is most common in northern India but is also found form northern Africa to central Asia. Guggul comprises a yellowish resin produced by the stem of the plant that has been used for more than 2000 years as part of India's traditional medicine known as Ayurveda. Guggul has long been known to have anti-lipedemic effects. Guggulsterone isolated from guggul has been identified as the bioactive constituent responsible for guggul's therapeutic effects. Guggul lowers serum triglycerides as well as LDL and VLDL cholesterols, and it raises HDL cholesterol [12, 13]. Mechanistically, the stereoisomers E- and Z-guggulsterone have been identified as the active ingredients by acting as antagonist ligands for the bile acid receptor farnesoid X receptor (FXR), which regulates cholesterol homeostasis [14]. Additionally, guggul is an antioxidant that prevents the oxidation of LDL and is therefore beneficial against atherosclerosis [15]. In one published study, guggul was administered to dogs and monkeys in combination with garlic powder, which demonstrated a statistically significant lowering of blood cholesterol [16].

In another embodiment, garlic is administered to a mammal in need of a cholesterol-lowering agent along with a composition of ingredients comprising policosanol, niacin, guggul, cynara scolymus, red yeast rice, ginger, holy basil, L-carnitine, chromium picolinate, coenzyme Q10, pantothenic acid, grape seed extract, mormodica charantia and garcinia indica. It is known in the art that garlic contains various antioxidant phytochemicals that prevent oxidative damage caused by free radical stress. Garlic is known to inhibit platelet aggregation, reduce blood pressure, and to possess cardioprotective properties. A disease-relevant antioxidant effect of garlic was demonstrated in a double blind, randomized trial in 65 intermediate risk patients (age 60+/−9 years, 79% male) who were treated with a placebo capsule or a capsule containing aged garlic extract (250 mg) plus Vitamin B12 (100 microg), folic acid (300 microg), Vitamin B6 (12.5 mg) and 1-arginine (100 mg) given daily for a year (Budoff et al. 2009). At 1 year, atherosclerosis as detected by coronary artery calcium scanning progression was significantly lower in the garlic and antioxidant group compared to the placebo group after adjustment of cardiovascular risk factors (p<0.05). Moreover, total blood cholesterol, LDL, homocysteine, and apoB-immune complexes were decreased, whereas HDL, OxPL/apoB, and lipoprotein (a) were significantly increased in garlic plus antioxidant group compared to placebo [17]. Additionally, numerous studies have demonstrated that garlic and aged garlic extract are capable of reducing oxidization of LDL. In a published meta-analysis of 952 subjects from 16 trials, various garlic preparations were found to induce a 12% lowering of total cholesterol compared with placebo controls [18]. The reductions in plasma cholesterol were evident one month after therapy and persisted for at least six months. Studies of Allicor, a time-release garlic tablet, have proven success of this drug at lowering cholesterol and improving clinical cardiac parameters. The AMAR (Atherosclerosis Monitoring and Atherogenicity Reduction) study examined the effect of two-year treatment with Allicor on the progression of atherosclerosis. This double-blinded placebo-controlled randomizes study recruited 196 asymptomatic men aged 40-74 and measured the carotid-intima-media thickness using high-resolution B-mode ultrasonography. Allicor treatment significantly reduced the progression of atherosclerosis compared to the placebo group (p=0.002) [19]. In one study of Allicor conducted in 51 patients with coronary heart disease, 12-month treatment decreased the cardiovascular risk by 1.5-fold in men and by 1.3-fold in women [20]. Another published meta-analysis that analyzed the results from 26 studies concluded that garlic powder and aged garlic extract had a significant impact in lowering serum cholesterol levels in patients, whereas garlic oil was effective for lowering serum triglyceries [21]. Thus, each preparation may have distinct effects on serum lipids; however, each of these distinct forms of garlic would be beneficial to patients with cardiovascular disease.

In animal studies investigating the effects of garlic on the complications associated with high cholesterol, supplementation of a cholesterol-rich diet with garlic oil was found to have protective effects against apoptosis of cardiac cells [22]. In a mouse model, garlic supplementation was found to have protective effects against fatty liver disease [23]. In this study, mice were given an alcohol liquid diet to induce fatty liver disease, and treatment with garlic oil caused a normalization of serum aminotransferase levels and liver antioxidant enzymes and reduced levels of blood cholesterol. In an animal study evaluating the influence of garlic on diabetic complications, mice were injected with streptozotocin to induce diabetes and treated with garlic bulb for 28 consecutive days. Garlic had a beneficial effect in alleviating signs of metabolic syndrome (hyperglycemia and hyperlipidemia) and reduced cellular toxicity in mice [24]. Hence, the studies in animals validate that garlic has effects in rescuing cells and tissues from the effects of high cholesterol.

In another embodiment, cynara scolymus is administered as a cholesterol-lowering agent along with a composition of ingredients comprising policosanol, niacin, guggul, garlic, red yeast rice, ginger, holy basil, L-carnitine, chromium picolinate, coenzyme Q10, pantothenic acid, grape seed extract, mormodica charantia and garcinia indica. Cynara scolymus, or artichoke leaf extract, is a Mediterranean thistle-like plant that has been widely cultivated for embittering alcoholic and soft drinks and for preparation of herbal teas and herbal medicinal products. In the art of herbal medicine, artichoke has been used traditionally for gallbladder problems, high cholesterol, and digestive liver disorders. In a randomized double blind placebo controlled study of 131 adults, a commercial preparation of artichoke leaf extracts was administered to patients daily for 12 weeks [25]. The results showed that total plasma cholesterol decreased by an average of 4.2% in the treated group and increased by an average of 1.9% in the placebo group (p=0.025 for the difference between the treated and placebo groups). A study performed in vitro demonstrated that cynaroside is the main active ingredient within artichoke extract that is responsible for inhibiting hepatic cholesterol biosynthesis [26]. There is also supporting evidence from animal studies for the effects of cynara scolymus on cholesterol metabolism. In one example where rats fed a high cholesterol diet were also given artichoke leaf extract (1.5 g/kg/day) by gavage for 2 weeks, the data showed that oxidative stress was reduced and lipid levels were lowered in the plasma [27]. In relation to the reduction of oxidative stress mediated by artichoke extract, there is also evidence for improved endothelial function in patients. In 18 patients with moderately elevated lipids, a dose of 20 ml/die of artichoke juice for six weeks revealed reductions in VCAM and ICAM molecules in the blood and an increased in brachial flow-mediated vasodilation [28]. Notably, there was a reduction in total cholesterol and LDL cholesterol in these patients, although the concentrations of triglycerides were increased in patients who consumed artichoke juice. Cumulatively, these changes in serum markers suggest that artichoke juice has a positive effect on endothelial cell function in patients with elevated blood cholesterol.

In another embodiment, red yeast rice is administered as a cholesterol-lowering agent along with a composition of ingredients comprising policosanol, niacin, guggul, cynara scolymus, garlic, ginger, holy basil, L-carnitine, chromium picolinate, coenzyme Q10, pantothenic acid, grape seed extract, mormodica charantia and garcinia indica. As implied by its name, red yeast rice, a substance that is extracted from rice that has been fermented with the yeast Monascus purpureus. Red yeast rice has been used throughout Asia for centuries as a traditional medicine. Lovastatin, a member of the drug class of statins, is a naturally occurring product found in foods such as red yeast rice and oyster mushrooms, that is approved for the treatment of high cholesterol in the patented prescription drug Mevacor. Red yeast rice exerts cholesterol-lowering effects similar to those of statins without the side effects that are associated with prescription drugs. Typical doses of red yeast rice are up to 2 g per day, although traditional Chinese medicine uses much higher doses. Red yeast rice can be administered in a dried powdered form (also known as Zhi Tai) and extracted with alcohol (known as Xue Zhi Kang).

There is an abundance of clinical evidence to support the use of red yeast rice as a dietary supplement for controlling blood cholesterol levels. In a published paper from 2010, patients who had experienced myalgias, gastrointestinal intolerance or elevated alanine aminotransferase (indicative of damage to the liver) while on statin drugs were evaluated for their response to treatment with red yeast rice for at least four weeks [29]. Significantly, red yeast rice consumption resulted lowered total cholesterol by 13% and LDL cholesterol by 19%. In another published example, 4 HypoCol capsules (100% red yeast rice, 600 mg/capsule) were administered to patients to evaluate the effects of red yeast rice on plasma lipids [30]. In this randomized, double blind placebo controlled study, patients receiving HypoCol capsules showed a 15.5% reduction in total cholesterol and a 23% reduction in LDL cholesterol. Similarly, children with Familial Hypercholesteorlemia and Familial Combined Hyperlipidemia aged 8-16 years were safely treated with a daily dietary supplement consisting of 200 mg of red yeast rice extract administered for 8 weeks and achieved significantly lower blood cholesterol levels [8]. Red yeast rice has also been combined with other natural ingredients to improve dyslipidemia in patients. For example, LDL cholesterol was significantly reduced in patients given red yeast rice extract together with plant stanols and sterols [31]. Interestingly, these investigators also comment that the results achieved with their study surpass the LDL-lowering effects that have been observed with statin medications.

In another embodiment, ginger is administered as a cholesterol-lowering agent along with a combination of ingredients comprising policosanol, niacin, guggul, cynara scolymus, garlic, red yeast rice, holy basil, L-carnitine, chromium picolinate, coenzyme Q10, pantothenic acid, grape seed extract, mormodica charantia and garcinia indica. Ginger, the rhizome of the plant Zingiber officinale, is known in the art to possess hypolipidemic and anti-oxidant properties and its uses date back to ancient China and India. In a published study conducted in apolipoprotein E-deficient mice that are prone to atherosclerosis, the animals were divided into 3 groups: those that received 25 mg ginger extract, those that received 250 mg ginger extract, and those that received placebo treatment for 10 weeks in their drinking water [32]. The study results showed a 44% reduction of atherosclerotic lesions in mice that received the high dose of ginger extract. Moreover, a 76% reduction in cholesterol biosynthesis by macrophages was observed in the group that received the high dose. In both the high and low-dose treatment groups, there was reduced oxidation of LDL. As LDL becomes oxidized, it can product inflammation in arteries and is therefore a hallmark of the atherosclerotic process. Thus, one mechanism of action of ginger is in reducing the basal oxidative state.

Studies have also been performed that support the use of ginger as an adjunct to statin drugs to treat elevated blood cholesterol. In an animal study, rats were randomized into several groups: ginger extract (400 mg/kg), atorvastatin (Lipitor; 20 mg/kg) alone or with ginger extract or vitamin E, and atorvastatin (80 mg/kg) alone or with ginger extract or vitamin E [33]. After 4 weeks, atorvastatin at the high (80 mg/kg) dose induced hepatotoxicity when given alone, although serum cholesterol was lowered. Significantly, co-treatment with ginger extract reduced the liver lesions induced by the drug while also lowering serum cholesterol. Similarly, administration of ginger extract alleviated the hepatotoxic effects of acetaminophen in a rat model [34]. A single dose of ginger extract (200 and 400 mg/kg, p.o.) prior to administration of acetaminophen reduced the concentrations of serum markers of liver damage (transaminases and alkaline phosphatase). These data support using ginger extract and reduced doses of statins to minimize adverse side effects in patients undergoing treatment for hypercholesteremia.

In another embodiment, holy basil (Ocimum sanctum) is administered as a cholesterol-lowering agent along along with a combination of ingredients comprising policosanol, niacin, guggul, garlic, cynara scolymus, red yeast rice, ginger, L-carnitine, chromium picolinate, coenzyme Q10, pantothenic acid, grape seed extract, mormodica charantia and garcinia indica. Holy basil is an herb native to Indie that represents an important plant in Ayurvedic medicine. Holy basil has been used for centuries to treat a range of conditions, including heart problems, arthritis, asthma, and bronchitis. Holy basil (or tulsi, as it is known in India) is considered an “adaptogenic herb”, referring to its use for increasing the body's resistance to physical, chemical or environmental stress. Adaptogens are so named based on their ability to correct the body's state of imbalance. One well-established effect of holy basil is for modulating elevated cholesterol in the blood and preventing its build up in the arteries. Holy basil has been tested in a randomized, placebo-controlled, crossover single blind trial in patients with diabetes mellitus, which revealed significant drop in fasting and postprandial blood glucose and a reduction in blood cholesterol levels [35]. An active compound from holy basil has been identified as tetracyclic triterpenoid, which has anti-diabetic and cholesterol-lowering effects [36]. Another study identified the oils within the leaves of holy basil as having lipid-lowering and anti-oxidant actions [37, 38]

In another embodiment, L-carnitine is administered as a cholesterol-lowering agent along with a combination of ingredients comprising policosanol, niacin, guggul, garlic, cynara scolymus, red yeast rice, ginger, holy basil, chromium picolinate, coenzyme Q10, pantothenic acid, grape seed extract, mormodica charantia, and garcinia indica. L-carnitine is a naturally-occuring amino acid that serves as a transporter of fatty acids in the mitochondria of cells. Owing to this role in lipid metabolism, L-carnitine supplementation is useful for lowering the levels of oxidized LDL in the body. In type 2 diabetes, are under high levels of oxidative stress, which is associated with elevated levels of oxidized LDL that carry a cardiovascular risk. Oxidation of LDL leads to increased LDL uptake by cells in the arterial walls, leading to the formation of atherosclerotic plaques [39]. Significantly, a recent meta-analysis of the available data on Pubmed revealed that supplementation with L-carnitine leads to lowering of total cholesterol and LDL concentrations in blood in patients with type 2 diabetes [40]. In a rat model, supplementation with 400 mg/kg/day of L-carnitine reversed the effects of a hypertensive agent and reversed its effects on cardiac fibrosis [41]. Hence, L-carnitine supplementation can protect the blood vessels and heart from damage.

In another embodiment, chromium picolinate is administered as a cholesterol-lowering agent along with a combination of ingredients comprising policosanol, niacin, guggul, garlic, cynara scolymus, red yeast rice, ginger, holy basil, L-carnitine, coenzyme Q10, pantothenic acid, grape seed extract, mormodica charantia, and garcinia indica. Chromium picolinate is a nutritional supplement that has been included in weight loss supplements due to its effects in improving carbohydrate and fat metabolism. Chromium is a trace mineral that is known in the art to be important for the maintenance of healthy carbohydrate and lipid levels [42]. Deficiency of chromium can result from dietary insufficiency as chromium is found in brewer's yeast, onions, tomatoes, whole grains, brain cereals, and oysters. Dietary deficiency in chromium is believed to be widespread owing to food processing methods that remove chromium from foods. An example, the chromium contained in germ and bran is removed from whole grains during the milling process that is used to make flour. Individuals with diabetes or heart disease may also have higher requirements for chromium that is not being met due to diets high in processed foods. Picolinic acid is a metabolite of the amino acid tryptophan that forms stable complexes with metal ions, thereby forming biologically active chromium picolinate and improving the bioavailability of chromium.

Clinical evidence exists that chromium picolinate is useful for treating lipid disorders. In a double-blind crossover study, 28 volunteers were given gelatin capsules containing either chromium picolinate (1.6 mg containing 200 μg Cr³⁺) mixed with 5 mg calcium phosphate or placebo containing only 5 mg calcium phosphate [43]. The capsules were administered daily for two 42-day periods with a 14-day period off capsules between treatments. The concentrations of total blood cholesterol, LDL cholesterol, and apoplipoprotein B (contained in the LDL fraction) were significantly reduced by treatment with chromium picolinate. Specifically, the results revealed a 7% decrease in total cholesterol (276 to 256 mg per dl) and LDL cholesterol decreased 10.5% (200 to 178 mg per dl) in response to chromium picolinate supplementation. Hence, the investigators concluded that chromium picolinate could be used along with dietary modifications and/or lipid-reducing drugs for the treatment and prevention of heart disease.

In humans, chromium picolinate (1000 mug/day) therapy was shown to improve insulin resistance in HIV-positive subjects. Eight subjects on antiretroviral therapy were treated with chromium picolinate for eight weeks and insulin sensitivity was measured with a hyperinsulinaemic-euglycaemic insulin clamp [44]. The mean rate of glucose disposal was measured to be 4.41 mg glucose/kg lean body mass (LBM)/min (range 2.67-5.50), which increased to 6.51 mg/kg LBM/min (range 3.19-12.78, p=0.03), an increase of 25% following chromium picolinate treatment. In another study, 46 HIV-positive patients with elevated glucose and/or lipids and who were insulin-resistant were treated with 400 micrograms/day of chromium nicotinate or placebo for 16 weeks [45]. Chromium treatment resulted in significant decreases in insulin (pre-treatment: 102 (85-226); post-treatment: 99 (59-131) pmol/L, p=0.003), triglycerides, total body fat mass (mean+/−SEM) (pre: 17.3+/−1.7; post: 16.3+/−1.7 kg; p=0.002) and trunk fat mass (pre: 23.8+/−1.9; post: 22.7+/−2.0%; p=0.008).

There is also scientific evidence documenting the mechanisms of action of chromium picolinate on carbohydrate and lipid metabolism in rats treated with streptozotocin to induce diabetes. In a study where chromium picolinate was administered to diabetic rats at low (human equivalent) and high doses (2.90 and 13.20 μg Cr kg(−1) day(−1), respectively, elevated levels of hepatic and cerebral free fatty acids and malondialdehyde were significantly reduced by low doses of chromium picolinate and were nearly normalized to the levels of non-diabetic rats in the high dose group [46]. There is also evidence from this rat model that chromium picolinate can protect against microvascular organ damage that is associated with metabolic disorders. In streptozotocin-treated rats that were fed a high-fat diet, supplementation with chromium picolinate (80 μg/kg body weight daily for two weeks) lowered blood glucose by an average of 63% (P<0.001), total cholesterol by 9.7% (P<0.001), and triglycerides by 6.6% (P<0.001) compared with the group that received a high-fat diet alone. Moreover, chromium picolinate also normalized the histological appearance of the kidneys and liver, suggesting that chromium repairs the microvascular complications that are associated with a high fat diet [47]

In another embodiment, coenzyme Q10 is administered as a cholesterol-lowering agent along with a combination of ingredients comprising policosanol, niacin, guggul, garlic, cynara scolymus, red yeast rice, ginger, holy basil, L-carnitine, chromium picolinate, pantothenic acid, grape seed extract, mormodica charantia, and garcinia indica. Coenzyme Q10 is a naturally occurring compound found in nearly every cell in the body that plays a key role in producing energy in the mitochondria in the form of ATP. In each human cell, food energy is converted into energy in the mitochondria with the aid of coenzyme Q10. Ninety-five percent of all the human body's energy requirements (ATP) are converted with the aid of coenzyme Q10. Therefore, those organs with the highest energy requirements, such as the heart, the lungs, and the liver have the highest coenzyme Q10 concentrations.

With respect to the present invention, coezyme Q10 has been demonstrated to have beneficial effects on cholesterol metabolism and its associated cardiovascular complications. Lee et al. showed that subjects with higher coenzyme Q10 concentrations (at least 516.0 nmol/L) had lower risks of coronary artery disease [48]. In children, it has also been reported that an increase in the ratios of total cholesterol to coenzyme Q10 is associated with obesity and might therefore be useful to predict medical complications [49]. In another study, coenzyme Q10 supplements that were administered for 12 weeks at a dose of 150 mg/d to patients with coronary artery disease were found to increase the levels of antioxidant enzyme activity in the blood [50]. Moreover, studies related to cholesterol levels with coenzyme Q10 supplements have been performed. For example, in one publication, stain-treated patients with type 2 diabetes were randomized into groups that received oral coenzyme Q10 (200 mg/day) or placebo for 12 weeks [51]. The results showed that coenzyme Q10 increased brachial artery flow-mediated dilation, indicative of improved endothelial function. Hence, these lines of evidence support the use of coenzyme Q10 as a dietary supplement to address high cholesterol as well as the associated complications.

In another embodiment, pantothenic acid is administered as a cholesterol-lowering agent along with a combination of ingredients comprising policosanol, niacin, guggul, garlic, cynara scolymus, red yeast rice, ginger, holy basil, L-carnitine, chromium picolinate, coenzyme Q10, grape seed extract, mormodica charantia, and garcinia indica. Pantothenic acid, also referred to as pantothenate or vitamin B5, is a component of coenzyme A, which carries out acetylation reactions within cells. Synthesis of hormones and fats as well as essential cellular processes such as cell division and cell signaling are dependent upon the activity of coenzyme A. Pantothenic acid is present in a variety of food sources, including cheese, peas, lean meat, poultry, fish, and whole grain cereals. Several studies have indicated that pantothenic acid can help to reduce LDL cholesterol or triglycerides in the blood. For example, in a published study using a mouse model, the hypolipidemic effects of pantothenic acid were demonstrated by administration of 150 mg/kg body weight phosphopantothenate for 10 days [52]. In this study, phosphopantothenate treatment decreased the content of triglycerides, total cholesterol and cholesterol esters in serum and adipose tissue. It is known in the art that pantethine, a derivative of pantothenic acid, is often administered at doses of 300 mg taken several times daily to lower blood cholesterol concentrations in humans.

In another embodiment, grape seed extract is administered as a cholesterol-lowering agent along with a combination of ingredients comprising policosanol, niacin, guggul, garlic, cynara scolymus, red yeast rice, ginger, holy basil, L-carnitine, chromium picolinate, coenzyme Q10, pantothenic acid, mormodica charantia, and garcinia indica. Grape seed extract is known in the art to be enriched for potent antioxidants including polyphenols, flavenoids and vitamin E. It is known in the art that each of these antioxidants can lower the levels of oxidized LDL cholesterol through their effects in inhibiting oxidation reactions. The oxidized form of LDL is particularly harmful because it readily attaches to epithelial cells in the arteries, which mediates adhesion of inflammatory cells to the vessel walls. In modern diets that are enriched for processed foods, grape seed extract has been used to reverse the hyperglycemic and hyperlipidemic effects. For example, in a recent study, 52 mildly hyperlipidemic patients were divided into two groups: one group received 200 mg/day grape seed extract and the other group received placebo [53]. Grape seed extract consumption reduced total cholesterol (p=0.015), LDL cholesterol (p=0.014), and oxidized LDL cholesterol (p=0.008) concentrations in the serum. In another study, 32 patients with type 2 diabete mellitus were randomized into groups that received either grape seed extract (600 mg/day) or placebo for 4 weeks for assessment of cardiovascular parameters [54]. The expression of inflammatory and glycemic markers was reduced in blood in grape seed extract vs. placebo treatment groups. The levels of serum cholesterol were also reduced significantly (p=0.05). Supplementation with grape seed extract can therefore modulate blood cholesterol levels that are associated with pathological conditions such as type 2 diabetes.

In another embodiment, momordica charanatia is administered as a cholesterol-lowering agent along with a combination of ingredients comprising policosanol, niacin, guggul, garlic, cynara scolymus, red yeast rice, ginger, holy basil, L-carnitine, chromium picolinate, coenzyme Q10, pantothenic acid, grape seed extract, and garcinia indica. Originating in tropical and subtropical climates including parts of the Amazon, east Africa, Asia, and the Carribean, this vegetable (also known as bitter melon) has been used in traditional medicine for the treatment of type 2 diabetes as well as a plethora of other conditions. Clinical and experimental studies also support the use of momoridca charantia for lowering plasma cholesterol levels. Mormorica charantia has been administered in capsules, as component of fruit juice, and subcutaneously.

To date, approximately 100 in vivo studies have documented that momoridca charantia lowers blood sugar levels and reduces cholesterol levels, thereby serving as a nutritional supplement to address metabolic and lipid disorders. In one study, patients with type 2 diabetes were given bitter melon in liquid form (5.56 ml/24 h), resulting in reductions in serum glucose levels (93.7+/−9.63 vs. 88.35+/−6.31 mg/dl, p=0.78) and lowering of plasma cholesterol concentrations (192+/−14.23 vs. 170.6+/−15.1 mg/dl, p<0.03) [55]. In a study that was conducted in rats fed cholesterol-enriched diets, momoridca charantia administered as a freeze-dried powder for two weeks raised the concentrations of HDL cholesterol in the serum, suggestive of an anti-atherogenic effect [56]. Moreover, momordica charantia reduced hepatic total cholesterol and triglyceride levels in rats fed both cholesterol-free and cholesterol-enriched diets. This compound is therefore useful as a supplement for addressing both dietary and endogenous cholesterol synthesis in the liver.

The composition of said nutraceutical formulation contains garcinia indica, a polygamodioecious tree belonging to the Clusiaceae family that is native to tropical Asia, Africa, Polynesia and in humid forests of Western Ghats of South India and the North Eastern states of India. The fruit extract form garcinia indica contains hydoxycritic acid, which has been shown to have cholesterol-lowering and anti-obesity effects [57]. Garcinol is a polyisoprenylated benzophenone that is extracted from the rind of the fruit of Garcinia indica, which exhibits potent anti-oxidant activity [58]. The ability of garcinol to inhibit oxidative stress, thereby reducing cellular damage and inflammation, is relevant to the formation of oxidized LDL cholesterol, a major component in the formation of atherosclerotic plaques.

The herb-based composition of the present invention can be used in beverages, tonics, infusions, or food-stuffs alone, or in combination with other dietary supplements or therapeutics that are known in the art. The herb-based composition of the present invention can be used alone or further formulated with pharmaceutically acceptable compounds, vehicles, or adjuvants with a favorable delivery profile, i.e., suitable for delivery to a subject. A composition of the present invention may be formulated to be compatible with its intended route of administration; for example (but not limited to) oral compositions, which generally include an inert diluent or an edible carrier. Oral compositions can be enclosed in numerous delivery vehicles known in the art, including gelatin capsules, tablets, reconstitutable powders, lozenges, liquids, suspensions, emulsions, capsules, and combinations thereof.

Having generally described this technology, a further understanding can be obtained by reference to specific examples, which are provided herein for purposes of illustration only, and are not intended to be limiting.

### EXAMPLES

Nutraceutical-grade tablets were manufactured, each containing the following ingredients: 5.16 mg of policosanol, 53.57 mg of niacin, 62.5 mg of guggul, 37.5 mg of garlic, 250 mg of cynara scolymus, 150 mg of red yeast rice, 75 mg of ginger, 125 mg of holy basil, 186.57 mg of L-carnitine, 0.42 mg of chromium picolinate, 38.27 mg of coenzyme Q10, 148.81 mg of pantothenic acid, 50 mg of grape seed extract, 125 mg of momordica charantia, and 75 mg of garcinia indica. Two tablets were ingested orally twice per day (total of 4 tablets per day), and cholesterol concentrations in blood were determined using lipid panel tests known in the art.

The results for a representative human subject who ingested said combination of ingredients are summarized in Table 1. This subject was a 38-year old male who initially presented with borderline high levels of total cholesterol, LDL cholesterol, and triglycerides, and who ingested the tablets at the indicated dosage daily for 30 days. This patient reported that no additional cholesterol-modifying agents were being taken and no dietary changes were implemented during the test period. Table 1 presents the “baseline” cholesterol levels (prior to ingestion of the nutraceutical formulation) and “after treatment” (after 30 days of daily intake). The results show that the nutraceutical formulation affords beneficial effects to the individual in the lipid panel test; namely, reduced total cholesterol, reduced LDL cholesterol, reduced triglycerides, and increased HDL cholesterol on day 30 of treatment.

TABLE 1
Measurement of blood cholesterol in response to oral
ingestion of a nutraceutical formulation (results in mg/dl)

| | Total | | | |
|---|---|---|---|---|
| | Cholesterol | LDL | HDL | Triglycerides |
| Baseline | 220 | 145 | 40 | 173 |
| After treatment | 188 | 119 | 58 | 147 |

Similar beneficial cholesterol-lowering effects of this nutraceutical formulation have been observed in other subjects as well. Following administration of the tablets described above, a 69-year old male and a 45 year-old female presented with 25% and 20% reductions in LDL cholesterol, respectively, following 120 consecutive days of treatment with this nutraceutical formulation. The positive response of these patients to this nutraceutical formulation constitutes direct evidence that this combination of ingredients is effective in lowering the harmful levels of blood cholesterol.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

### REFERENCES

1. Daniels, T. F., et al., Lipoproteins, cholesterol homeostasis and cardiac health. Int J Biol Sci, 2009. 5 (5): p. 474-88.
2. Businaro, R., et al., Cellular and molecular players in the atherosclerotic plaque progression. Ann N Y Acad Sci, 2012. 1262: p. 134-41.
3. Superko, H. R. and R. R. Gadesam, Is it LDL particle size or number that correlates with risk for cardiovascular disease? Curr Atheroscler Rep, 2008. 10 (5): p. 377-85.
4. Calpe-Berdiel, L., J. C. Escola-Gil, and F. Blanco-Vaca, New insights into the molecular actions of plant sterols and stanols in cholesterol metabolism. Atherosclerosis, 2009. 203 (1): p. 18-31.
5. Kamal-Eldin, A. and A. Moazzami, Plant sterols and stanols as cholesterol-lowering ingredients in functional foods. Recent Pat Food Nutr Agric, 2009. 1 (1): p. 1-14.
6. Wang, L. J. and B. L. Song, Niemann-Pick C1-Like 1 and cholesterol uptake. Biochim Biophys Acta, 2012. 1821 (7): p. 964-72.
7. Chen, J. T., et al., Meta-analysis of natural therapies for hyperlipidemia: plant sterols and stanols versus policosanol. Pharmacotherapy, 2005. 25 (2): p. 171-83.
8. Guardamagna, O., et al., The treatment of hypercholesterolemic children: efficacy and safety of a combination of red yeast rice extract and policosanols. Nutr Metab Cardiovasc Dis, 2011. 21 (6): p. 424-9.
9. Gille, A., et al., Nicotinic acid: pharmacological effects and mechanisms of action. Annu Rev Pharmacol Toxicol, 2008. 48: p. 79-106.
10. Lavigne, P. M. and R. H. Karas, The current state of niacin in cardiovascular disease prevention:

a systematic review and meta-regression. J Am Coll Cardiol, 2013. 61 (4): p. 440-6.
11. Philpott, A. C., et al., Niacin improves lipid profile but not endothelial function in patients with coronary artery disease on high dose statin therapy. Atherosclerosis, 2013. 226 (2): p. 453-8.
12. Nityanand, S. and N. K. Kapoor, Hypocholesterolemic effect of Commiphora mukul resin (guggal). Indian J Exp Biol, 1971. 9 (3): p. 376-7.
13. Nityanand, S. and N. K. Kapoor, Cholesterol lowering activity of the various fractions of the guggal. Indian J Exp Biol, 1973. 11 (5): p. 395-6.
14. Urizar, N. L. and D. D. Moore, GUGULIPID: a natural cholesterol-lowering agent. Annu Rev Nutr, 2003. 23: p. 303-13.
15. Wang, X., et al., The hypolipidemic natural product Commiphora mukul and its component guggulsterone inhibit oxidative modification of LDL. Atherosclerosis, 2004. 172 (2): p. 239-46.
16. Dixit, V. P., et al., Hypolipidemic activity of guggal resin (Commiphora mukul) and garlic (Alium sativum linn.) in dogs (Canis familiaris) and monkeys (Presbytis entellus entellus Dufresne). Biochem Exp Biol, 1980. 16 (4): p. 421-4.
17. Budoff, M. J., et al., Aged garlic extract supplemented with B vitamins, folic acid and L-arginine retards the progression of subclinical atherosclerosis: a randomized clinical trial. Preventive medicine, 2009. 49 (2-3): p. 101-7.
18. Silagy, C. and A. Neil, Garlic as a lipid lowering agent—a meta-analysis. J R Coll Physicians Lond, 1994. 28 (1): p. 39-45.
19. Orekhov, A. N., et al., Anti-Atherosclerotic Therapy Based on Botanicals. Recent Pat Cardiovasc Drug Discov, 2012.
20. Sobenin, I. A., et al., The effects of time-released garlic powder tablets on multifunctional cardiovascular risk in patients with coronary artery disease. Lipids Health Dis, 2010. 9: p. 119.
21. Zeng, T., et al., A meta-analysis of randomized, double-blind, placebo-controlled trials for the effects of garlic on serum lipid profiles. J Sci Food Agric, 2012. 92 (9): p. 1892-902.
22. Cheng, Y. C., et al., Garlic oil attenuates the cardiac apoptosis in hamster-fed with hypercholesterol diet. Food Chem, 2013. 136 (3-4): p. 1296-302.
23. Raghu, R., et al., Transcriptome analysis of garlic-induced hepatoprotection against alcoholic fatty liver. J Agric Food Chem, 2012. 60 (44): p. 11104-19.
24. Madkor, H. R., S. W. Mansour, and G. Ramadan, Modulatory effects of garlic, ginger, turmeric and their mixture on hyperglycaemia, dyslipidaemia and oxidative stress in streptozotocin-nicotinamide diabetic rats. Br J Nutr, 2011. 105 (8): p. 1210-7.
25. Bundy, R., et al., Artichoke leaf extract (Cynara scolymus) reduces plasma cholesterol in otherwise healthy hypercholesterolemic adults: a randomized, double blind placebo controlled trial. Phytomedicine, 2008. 15 (9): p. 668-75.
26. Gebhardt, R., Inhibition of cholesterol biosynthesis in primary cultured rat hepatocytes by artichoke (Cynara scolymus L.) extracts. J Pharmacol Exp Ther, 1998. 286 (3): p. 1122-8.
27. Kusku-Kiraz, Z., et al., Artichoke leaf extract reduces oxidative stress and lipoprotein dyshomeostasis in rats fed on high cholesterol diet. Phytother Res, 2010. 24 (4): p. 565-70.
28. Lupattelli, G., et al., Artichoke juice improves endothelial function in hyperlipemia. Life Sci, 2004. 76 (7): p. 775-82.
29. Venero, C. V., et al., Lipid-lowering efficacy of red yeast rice in a population intolerant to statins. Am J Cardiol, 2010. 105 (5): p. 664-6.
30. Bogsrud, M. P., et al., HypoCol (red yeast rice) lowers plasma cholesterol—a randomized placebo controlled study. Scand Cardiovasc J, 2010.44 (4): p. 197-200.
31. Feuerstein, J. S. and W. S. Bjerke, Powdered red yeast rice and plant stanols and sterols to lower cholesterol. J Diet Suppl, 2012. 9 (2): p. 110-5.
32. Fuhrman, B., et al., Ginger extract consumption reduces plasma cholesterol, inhibits LDL oxidation and attenuates development of atherosclerosis in atherosclerotic, apolipoprotein E-deficient mice. J Nutr, 2000. 130 (5): p. 1124-31.
33. Heeba, G. H. and M. I. Abd-Elghany, Effect of combined administration of ginger (Zingiber officinale Roscoe) and atorvastatin on the liver of rats. Phytomedicine, 2010. 17 (14): p. 1076-81.
34. Ajith, T. A., U. Hema, and M. S. Aswathy, Zingiber officinale Roscoe prevents acetaminophen-induced acute hepatotoxicity by enhancing hepatic antioxidant status. Food Chem Toxicol, 2007. 45 (11): p. 2267-72.
35. Agrawal, P., V. Rai, and R. B. Singh, Randomized placebo-controlled, single blind trial of holy basil leaves in patients with noninsulin-dependent diabetes mellitus. Int J Clin Pharmacol Ther, 1996. 34 (9): p. 406-9.
36. Patil, R., B. Ahirwar, and D. Ahirwar, Isolation and characterization of anti-diabetic component (bioactivity-guided fractionation) from Ocimum sanctum L. (Lamiaceae) aerial part. Asian Pac J Trop Med, 2011. 4 (4): p. 278-82.
37. Suanarunsawat, T., et al., Antioxidant Activity and Lipid-Lowering Effect of Essential Oils

Extracted from Ocimum sanctum L. Leaves in Rats Fed with a High Cholesterol Diet. J Clin Biochem Nutr, 2010. 46 (1): p. 52-9.
38. Suanarunsawat, T., et al., Anti-hyperlipidemic and cardioprotective effects of Ocimum sanctum L. fixed oil in rats fed a high fat diet. J Basic Clin Physiol Pharmacol, 2010. 21 (4): p. 387-400.
39. Steinberg, D., Low density lipoprotein oxidation and its pathobiological significance. J Biol Chem, 1997. 272 (34): p. 20963-6.
40. Vidal-Casariego, A., et al., Metabolic Effects of L-carnitine on Type 2 Diabetes Mellitus: Systematic Review and Meta-analysis. Exp Clin Endocrinol Diabetes, 2013.
41. Zambrano, S., et al., I-Carnitine protects against arterial hypertension-related cardiac fibrosis through modulation of PPAR-gamma expression. Biochem Pharmacol, 2013.
42. Power, M. and R. Pratley, Alternative and complementary treatments for metabolic syndrome. Curr Diab Rep, 2011. 11 (3): p. 173-8.
43. Press, R. I., J. Geller, and G. W. Evans, The effect of chromium picolinate on serum cholesterol and apolipoprotein fractions in human subjects. West J Med, 1990. 152 (1): p. 41-5.
44. Feiner, J. J., et al., Chromium picolinate for insulin resistance in subjects with HIV disease: a pilot study. Diabetes Obes Metab, 2008. 10 (2): p. 151-8.
45. Aghdassi, E., et al., In patients with HIV-infection, chromium supplementation improves insulin resistance and other metabolic abnormalities: a randomized, double-blind, placebo controlled trial. Curr HIV Res, 2010. 8 (2): p. 113-20.
46. Refaie, F. M., et al., Effect of chromium supplementation on the diabetes induced-oxidative stress in liver and brain of adult rats. Biometals, 2009. 22 (6): p. 1075-87.
47. Sahin, K., et al., Effect of chromium on carbohydrate and lipid metabolism in a rat model of type 2 diabetes mellitus: the fat-fed, streptozotocin-treated rat. Metabolism, 2007. 56(9): p. 1233-40.
48. Lee, B. J., et al., A significant correlation between the plasma levels of coenzyme Q10 and vitamin B-6 and a reduced risk of coronary artery disease. Nutr Res, 2012. 32 (10): p. 751-6.
49. Gvozdjakova, A., et al., Ratio of lipid parameters to coenzyme Q10 could be used as biomarker of the development of early complications of obesity in children. Bratisl Lek Listy, 2012. 113 (1): p. 21-5.
50. Lee, B. J., et al., Coenzyme Q10 supplementation reduces oxidative stress and increases antioxidant enzyme activity in patients with coronary artery disease. Nutrition, 2012. 28 (3): p. 250-5.
51. Hamilton, S. J., G. T. Chew, and G. F. Watts, Coenzyme Q10 improves endothelial dysfunction in statin-treated type 2 diabetic patients. Diabetes Care, 2009. 32 (5): p. 810-2.
52. Naruta, E. and V. Buko, Hypolipidemic effect of pantothenic acid derivatives in mice with hypothalamic obesity induced by aurothioglucose. Exp Toxicol Pathol, 2001. 53 (5): p. 393-8.
53. Razavi, S. M., et al., Red Grape Seed Extract Improves Lipid Profiles and Decreases Oxidized Low-Density Lipoprotein in Patients with Mild Hyperlipidemia. J Med Food, 2013.
54. Kar, P., et al., Effects of grape seed extract in Type 2 diabetic subjects at high cardiovascular risk: a double blind randomized placebo controlled trial examining metabolic markers, vascular tone, inflammation, oxidative stress and insulin sensitivity. Diabet Med, 2009. 26 (5): p. 526-31.
55. Inayat ur, R., et al., Serum sialic acid changes in non-insulin-dependant diabetes mellitus (NIDDM) patients following bitter melon (Momordica charantia) and rosiglitazone (Avandia) treatment. Phytomedicine, 2009. 16 (5): p. 401-5.
56. Jayasooriya, A. P., et al., Effects of Momordica charantia powder on serum glucose levels and various lipid parameters in rats fed with cholesterol free and cholesterol-enriched diets. J Ethnopharmacol, 2000. 72 (1-2): p. 331-6.
57. Padhye, S., et al., Emerging role of Garcinol, the antioxidant chalcone from Garcinia indica Choisy and its synthetic analogs. J Hematol Oncol, 2009. 2: p. 38.
58. Yamaguchi, F., et al., Antioxidative and anti-glycation activity of garcinol from Garcinia indica fruit rind. J Agric Food Chem, 2000. 48 (2): p. 180-5.

What is claimed:

1. A nutraceutical formulation for the prevention and management of elevated blood cholesterol comprising the following naturally occurring substances: policosanol, niacin, guggul, garlic, cynara scolymus, red yeast rice, ginger, holy basil, L-carnitine, chromium picolinate, coenzyme Q10, pantothenic acid, grape seed extract, momordica charantia, and garcinia indica.

2. The nutraceutical formulation of claim 1, wherein said combination of naturally occurring substances comprises the following: policosanol at a concentration between 5-500 mg, niacin at a concentration of between 50-500 mg, guggul at a concentration between 10-600 mg, garlic at a concentration between 3-300 mg, cynara scolymus at a concentration between 20-1000 mg, red yeast rice at a concentration between 10-1000 mg, ginger at a concentration between 10-700 mg, holy basil at a concentration between 10-500 mg, L-carnitine at a concentration between 10-500 mg, chromium picolinate at a concentration between 0.1-1.0 mg, coenzyme Q10 at a concentration between 30-300 mg, pantothenic acid at a concentration between 100-1000 mg, grape seed extract at a concentration between 5-1000 mg, momordica charantia at a concentration between 100-1000 mg, and garcinia indica at a concentration between 10-1000 mg.

3. The nutraceutical formulation of claim 1, wherein said combination of naturally occurring substances comprises the following: policosanol at approximately 5.16 mg, niacin at approximately 53.57 mg, guggul at approximately 62.5 mg, garlic at approximately 37.5 mg, cynara scolymus at approximately 250 mg, red yeast rice at approximately 150 mg, ginger at approximately 75 mg, holy basil at approximately 125 mg, L-carnitine at approximately 186.57 mg, chromium picolinate at approximately 0.42 mg, coenzyme Q10 at approximately 38.27 mg, pantothenic acid at approximately 148.81 mg, grape seed extract at approximately 50 mg, momordica charantia at approximately 125 mg, and garcinia indica at approximately 75 mg.

4. The nutraceutical formulation of claim 3, wherein said combination of ingredients is incorporated into tablets for administration by an oral route to a mammal in need of said formulation.

5. The nutraceutical formulation of claim 3, wherein said combination of ingredients is administered four times daily to a mammal in need of said formulation.

6. A method of lowering blood cholesterol in a mammal in need, comprising identifying a mammal suffering from high blood cholesterol and administering a combination of naturally occurring substances comprising: policosanol, niacin, guggul, garlic, cynara scolymus, red yeast rice, ginger, holy basil, L_carnitine, chromium picolinate, coenzyme Q10, pantothenic acid, grape seed extract, momordica charantia, and garcinia indica.

7. The method of claim 6, wherein said combination of naturally occurring substances is comprised of: policosanol at a concentration between 5-500 mg, niacin at a concentration of between 50-500 mg, guggul at a concentration between 10-600 mg, garlic at a concentration between 3-300 mg, cynara scolymus at a concentration between 20-1000 mg, red yeast rice at a concentration between 10-1000 mg, ginger at a concentration between 10-700 mg, holy basil at a concentration between 10-500 mg, L-carnitine at a concentration between 10-500 mg, chromium picolinate at a concentration between 0.1-1.0 mg, coenzyme Q10 at a concentration between 30-300 mg, pantothenic acid at a concentration between 100-1000 mg, grape seed extract at a concentration between 5-1000 mg, momordica charantia at a concentration between 100-1000 mg, and garcinia indica at a concentration between 10-1000 mg.

8. The method of claim 6, wherein said combination of naturally occurring substances is comprised of: policosanol at approximately 5.16 mg, niacin at approximately 53.57 mg, guggul at approximately 62.5 mg, garlic at approximately 37.5 mg, cynara scolymus at approximately 250 mg, red yeast rice at approximately 150 mg, ginger at approximately 75 mg, holy basil at approximately 125 mg, L-carnitine at approximately 186.57 mg, chromium picolinate at approximately 0.42 mg, coenzyme Q10 at approximately 38.27 mg, pantothenic acid at approximately 148.81 mg, grape seed extract at approximately 50 mg, momordica charantia at approximately 125 mg, and garcinia indica at approximately 75 mg.

9. The method of claim 8, wherein said combination of ingredients is incorporated into tablets for administration by an oral route to a mammal in need of said formulation.

10. The method of claim 8, wherein said combination of ingredients is administered four times daily to a mammal in need of said formulation.

11. The method of claim 6, wherein administration of said combination of ingredients to a mammal is indicated by one or more of the following: a) Total blood cholesterol concentrations; b) Low density lipoprotein concentrations in the blood; c) High density lipoprotein concentrations in the blood; d) Triglyceride concentrations in blood.

12. A method of treating individuals at risk for cardiovascular disease, comprising identifying an individual at risk for cardiovascular disease and administering a combination of naturally occurring substances comprising: policosanol, niacin, guggul, garlic, cynara scolymus, red yeast rice, ginger, holy basil, L carnitine, chromium picolinate, coenzyme Q10, pantothenic acid, grape seed extract, momordica charantia, and garcinia indica.

13. The method of claim 12, wherein cardiovascular risk is defined by one or more of the following: a) Dyslipidemia; b) Obesity; c) Smoking; d) High blood pressure; e) Diabetes; f) Insulin resistance; g) Hormone imbalance; h) Familial history of hypercholesterolemia and/or coronary artery disease.

14. The method of claim 12, wherein said combination of naturally occurring substances is comprised of: policosanol at a concentration between 5-500 mg, niacin at a concentration of between 50-500 mg, guggul at a concentration between 10-600 mg, garlic at a concentration between 3-300 mg, cynara scolymus at a concentration between 20-1000 mg, red yeast rice at a concentration between 10-1000 mg, ginger at a concentration between 10-700 mg, holy basil at a concentration between 10-500 mg, L-carnitine at a concentration between 10-500 mg, chromium picolinate at a concentration between 0.1-1.0 mg, coenzyme Q10 at a concentration between 30-300 mg, pantothenic acid at a concentration between 100-1000 mg, grape seed extract at a concentration between 5-1000 mg, momordica charantia at a concentration between 100-1000 mg, and garcinia indica at a concentration between 10-1000 mg.

15. The method of claim 12, wherein said combination of naturally occurring substances comprising: policosanol at approximately 5.16 mg, niacin at approximately 53.57 mg, guggul at approximately 62.5 mg, garlic at approximately 37.5 mg, cynara scolymus at approximately 250 mg, red yeast rice at approximately 150 mg, ginger at approximately 75 mg, holy basil at approximately 125 mg, L-carnitine at approximately 186.57 mg, chromium picolinate at approximately 0.42 mg, coenzyme Q10 at approximately 38.27 mg, pantothenic acid at approximately 148.81 mg, grape seed extract at approximately 50 mg, momordica charantia at approximately 125 mg, and garcinia indica at approximately 75 mg.

16. The method of claim 15, wherein said combination of ingredients is incorporated into tablets for administration by an oral route to a mammal in need of said formulation.

17. The method of claim 15, wherein said combination of ingredients is administered four times daily to a mammal in need of said formulation.
